(19) United States
(12) Patent Application Publication
(10) Pub. No.: US 2024/0225972 A9
(43) Pub. Date: Jul. 11, 2024

(54) A COMPOSITION FOR DEVELOPING HAIR COLOR COMPRISING POLYPHENOL

(71) Applicants: Korea Advanced Institute of Science and Technology, Daejeon (KR); MODAMODA CO., LTD., Seoul (KR)

(72) Inventors: Haeshin LEE, Seoul (KR); Daiheon LEE, Seoul (KR); Heungjin BAE, Seoul (KR)

(21) Appl. No.: 17/609,530

(22) PCT Filed: Jul. 16, 2021
(86) PCT No.: PCT/KR2021/009197

§ 371 Date: Nov. 8, 2021

(30) Foreign Application Priority Data
Jul. 8, 2021 (KR) 10-2021-0089820

(51) Int. Cl. A61K 8/34; A61K 8/365; A61K 8/368; A61K 8/41; A61K 8/84; A61K 8/92; A61Q 5/02; A61Q 5/10

(52) U.S. Cl. CPC A61K 8/347; A61K 8/365; A61K 8/368; A61K 8/415; A61K 8/84; A61K 8/922; A61Q 5/02; A61Q 5/10

(57) ABSTRACT

The present disclosure relates to a composition for developing hair color comprising polyphenol, a method of developing hair color using the composition, a shampoo for developing hair color comprising the composition, and a method of developing hair color using the composition. According to the present disclosure, the composition for developing hair color comprising trihydroxybenzene produces the development of hair color in brown, dark brown, or black and has excellent color development enhancing power, color development lasting power, and anti-gray hair, without using amine compounds, irritating alkaline agents, and oxidizing agents.

### TECHNICAL FIELD

The present disclosure relates to a composition for developing hair color comprising polyphenol, a shampoo for developing hair color comprising the composition, and a method of developing hair color using the composition.

### BACKGROUND

Hair color is determined by two types of melanin proteins present in the hair, one of which is eumelanin, which is involved in the chromaticity of hair from black to brown, and the other of which is phaeomelanin, which is involved in the chromaticity of hair from red to yellow. If these pigment proteins are insufficient, the hair becomes brown or gray, and the shape and size of melanin is the key to determining the hair color. The distribution density of these pigment molecules determines the contrast of hair color.

Generally, hair dyes consist of hydrogen peroxide, a pigment precursor, and ammonia solution. The ammonia component swells the hair or slightly peels off the hair surface cuticle, making it easier for the pigment precursor to penetrate into the hair. The hydrogen peroxide bleaches the melanin in the hair and at the same time reacts with the pigment precursor to dye the hair.

In the case of existing hair dyes, ammonia has a strong irritating odor and stimulates eyes or respiratory tract as well as the scalp, causing hypersensitivity reactions due to immune responses. Oxidizing agents such as hydrogen peroxide damage hair proteins and cuticles due to their strong oxidizing power, and are also irritating to the skin.

In order to overcome these disadvantages, a dye composition comprising a metal salt and a compound containing a catechol group such as dopamine has been suggested. In this case, the hair is dyed by an action mechanism in which dopamine acts as a ligand to form a complex with a metal ion of the metal salt. However, its downside is that a rather long dyeing procedure is required to secure excellent color development and metal ions may cause an allergic reaction to metal when in contact with the skin.

Under this background, the present inventors made intensive research efforts to develop a composition that colorizes hair differently from the existing dyeing principle of melanin oxidation and coloring of dyes in the hair cuticle, without using amine compounds, irritating alkaline agents, and oxidizing agents. As a result, the present inventors have completed the present disclosure by confirming that when a composition containing trihydroxybenzene is used, the compound is adsorbed to the hair surface and can deepen the hair color over time.

### SUMMARY

An object of the present disclosure is to provide a composition for developing hair color comprising trihydroxybenzene.

Another object of the present disclosure is to provide a method of developing hair color comprising the step of applying the composition for developing hair color to the hair.

Still another object of the present disclosure is to provide a shampoo for developing hair color comprising the composition for developing hair color.

Yet another object of the present disclosure is to provide a method of developing hair color comprising the step of cleansing the hair with the shampoo for developing hair color.

One aspect of the present disclosure for achieving the objects provides a composition for developing hair color comprising trihydroxybenzene.

Further, another aspect of the present disclosure for achieving the above object provides a method of developing hair color comprising the step of applying the composition for developing hair color to the hair.

### DESCRIPTION OF DRAWINGS

FIG. 1 shows the hair color developments of various polyphenol compounds.

FIG. 2 is a graph numerically showing a chromatic aberration for the hair color development effect of the inventive composition for developing hair color prepared in a shampoo formulation.

FIG. 3 shows the hair color development effects of the inventive composition for developing hair color prepared in a shampoo formulation.

Detailed Description The present disclosure will now be described in detail. Each description and embodiment disclosed in the present disclosure may be applied to other descriptions and embodiments, respectively. All of the combinations of various elements disclosed herein fall within the scope of the present disclosure. The scope of the present disclosure should not be interpreted to be limited by specific descriptions described below.

One aspect of the present disclosure for achieving the objects provides a composition for developing hair color comprising trihydroxybenzene.

As used herein, the term “developing hair color” indicates that a new color is expressed on hair's surface due to the adsorption of a composition containing a compound to the hair. Specifically, it includes making the hair black or darkening hair color from a light color to a dark color, or preventing hair whitening, such as gray hair. The ‘developing hair color’ has a mechanism different from the existing ‘hair dyeing.’ The principle of the existing hair dyeing is that the ammonia component of the hair dye swells the hair or excites the hair surface cuticle, and when the pigment precursor penetrates into the excited hair, an oxidizing agent such as hydrogen peroxide decolorizes the melanin in the hair and at the same time reacts with the pigment precursor to dye the hair. In the composition comprising trihydroxybenzene according to the present disclosure, however, the trihydroxybenzene is adsorbed to the hair surface and an oxidation reaction occurs due to a reaction with oxygen in the air over time, and the hair color changes to brown, dark brown, or black. Among various polyphenol compounds, we have confirmed that the composition comprising trihydroxybenzene had excellent hair color development.

The inventive composition for developing hair color is characterized in that it does not contain an amine compound, an oxidizing agent, and an irritating alkaline agent, so that the hair can be colored in dark color without damaging the hair.

The amine compound may be selected from the group consisting of polyethyleneimine, glycol chitosan, cystamine, pyrimidine, cytosine, thymine, uracil, 2,4,5,6-tetraaminopyrimidine, purine, adenine, guanine, hypoxanthine, xanthine, theobromine, caffeine, uric acid, and 2,6-diaminopurine, but not limited thereto.

The oxidizing agent may indicate an oxidizing agent other than atmospheric oxygen, and may include, for example, but is not limited to, hydrogen peroxide or urea peroxide.

The irritating alkaline agent may include, but is not limited to, ammonia or monoethanolamine.

From this point of view, the inventive composition for developing hair color may be for hair browning or blackening or for preventing whitening. Further, the inventive composition for developing hair color may enhance or sustain hair color development, and thus may be for enhancing hair color development or sustaining hair color development.

In the present disclosure, the trihydroxybenzene includes 1,2,4-trihydroxybenzene, 1,2,3-trihydroxybenzene and 1,3,5-trihydroxybenzene.

The composition for developing hair color includes 0.1 to 10% by weight of trihydroxybenzene, for example, 0.1 to 5% by weight, 0.1 to 3% by weight, 0.1 to 1% by weight, 1 to 10% by weight, 1 to 5% by weight, or 1 to 3% by weight, based on the total weight of the composition, but is not limited thereto.

The inventive composition for developing hair color may further include a mixture of Sesamum indicum (sesame) seed extract, Morus nigra fruit extract, Tuber melanosporum extract, Nigella sativa seed extract, Prunus serotina (wild cherry) fruit extract, and gallic acid. The weight ratio of the mixture and the trihydroxybenzene may be 1:0.1 to 3 by weight, for example, 1:0.1 to 1 by weight.

The mixture may be prepared, for example, in the following way: Sesamum indicum seed 250 g, Morus nigra fruit 50 g, Tuber melanosporum 50 g, Nigella sativa seed 50 g, and Prunus serotina (wild cherry) fruit 250 g are washed in purified water sufficiently; and they are extracted under reduced pressure at 65 to 90° C. for 6 to 8 hours using 20 L of purified water, respectively; and then, after obtaining hot water extracts, they are mixed with gallic acid to prepare the mixture. At this time, based on 100% by weight of the composition, 10% by weight of each hot water extract, 0.001% by weight of gallic acid, and the remaining purified water may be mixed.

The inventive composition for developing hair color may include at least one selected from the group consisting of caramel pigment, mulberry (Morus sp.) fruit extract, and green tea extract.

The inventive composition for developing hair color may be a shampoo, hair conditioner, hair treatment, hair lotion, hair gel, hair pack, hair cream, or hair essence formulation, but is not limited thereto.

Trihydroxybenzene contained in the inventive composition for developing hair color may remain in a trace amount on the hair surface even after washing the hair with water due to its adhesion/adsorption to proteins. In this way, when trihydroxybenzene present on the hair surface comes into contact with oxygen, an oxidation reaction occurs. As a result of the oxidation reaction, the hair gradually develops a dark color.

Further, another aspect of the present disclosure for achieving the above object provides a method of developing hair color comprising the step of applying the composition for developing hair color to the hair.

As used herein, the term “developing hair color” is as described above.

After applying the composition for developing hair color to the hair, the method may further include the step of leaving the composition for 1 to 30 minutes, for example, 1 to 5 minutes, or 1 to 3 minutes. Even for such a concise period of time of leaving, the hair can develop brown, dark brown, or black. The application method is not particularly limited. A method such as immersing the hair in the composition for developing hair color or spray-applying the composition for developing hair color to the hair may be used.

Further, another aspect of the present disclosure for achieving the above object provides a shampoo for developing hair color comprising the composition for developing hair color.

As used herein, the term “developing hair color” is as described above.

The shampoo for developing hair color may be one in which the aforementioned composition for developing hair color is added to a conventional shampoo composition. The amount of the composition for developing hair color is not particularly limited, but the composition for developing hair color may be added in an amount of 0.1 to 15% by weight or 1 to 15% by weight, based on the total weight of the shampoo for developing hair color.

The shampoo for developing hair color may further include any one or more selected from the group consisting of surfactants, pH adjusters, swelling agents, buffers, preservatives, fragrances, moisturizers, viscosity modifiers, aliphatic alcohols, and vitamins, but is not limited thereto.

The surfactant may be used without particular limitation as long as it is commonly used in the art and may be appropriately selected from among surfactants known in the art to achieve the effects of the present disclosure, for example, a cationic surfactant, an anionic surfactant, a nonionic surfactant, an amphoteric surfactant, or a mixture thereof.

More specifically, the surfactant may be alkyl sulfate (AS), alkyl ether sulfate (AES), sodium alkane sulfonate (SAS), alkyl trimethyl ammonium halide, alkyl pyridinium halide, alkyl imidazolium halide, polyethylene oxide-based surfactant, polyvinyl-based water-soluble polymer, and the like. As a non-limiting example, it may be any one or more selected from sodium dodecyl sulfate (SDS), sodium octylbenzene sulfonate (NaOBS), sodium dodecylbenzene sulfonate (SDBS), ammonium lauryl sulfate (ALS), Triton X-100, and polyvinylpyrrolidone (PVP), but is not limited thereto.

The pH adjuster is for adjusting the pH of the aqueous dye composition and may be used without particular limitation as long as it is commonly used in the art. It may be any one or more selected from sodium hydroxide, potassium hydroxide, triethanolamine, citric acid, and sodium citrate.

The swelling agent may be used without particular limitation as long as it is commonly used in the art. It may be any one or more selected from urea, thiourea, urea peroxide, polyoxymethylene urea, hydroxyethyl urea, ammonia, and salts thereof.

The buffer may be used without particular limitation as long as it is commonly used in the art. It may be any one or more selected from salicylic acid, sodium salicylate, lactic acid, sodium lactate, and the like.

The preservative may be used without particular limitation as long as it is commonly used in the art. It may be any one or more selected from methylparaben, propylparaben, phenoxyethanol, methyl p-hydroxybenzoate, and ethyl p-hydroxybenzoate, and the like. The fragrance may be used without particular limitation as long as it is commonly used in the art and may be selected from natural or artificial fragrances.

The moisturizer may be used without particular limitation as long as it is commonly used in the art, and commonly used components including glycols such as polyethylene glycols and oils may be used.

The viscosity modifier may be used without particular limitation as long as it is commonly used in the art and may be any one or more selected from poly stearate, sodium carboxymethyl cellulose, and the like. The aliphatic alcohol may be used without particular limitation as long as it is commonly used in the art and may be any one or more selected from an aliphatic alcohol having 16 to 22 carbon atoms, such as cetyl alcohol, stearyl alcohol, or behenyl alcohol. The vitamin may be used to supplement the nutrition of the hair.

Meanwhile, the shampoo for developing hair color according to the present disclosure may include water in addition to the above-mentioned components, and the water may be purified water such as ion-exchanged water or distilled water. In addition, the content is not particularly limited, but may be an amount capable of sufficiently dissolving or dispersing the above components.

In this way, adding the composition for developing hair color to a commonly used shampoo composition to prepare a shampoo for developing hair color makes it possible to develop a deep color of hair while shampooing every day. In the case of using the shampoo for developing hair color according to the present disclosure, hair color development occurs while trihydroxybenzene is adsorbed to the hair during the daily shampooing process, which causes additional hair color development instead of hair color loss, preventing hair color loss in the shampooing process, which occurs most commonly. Further, when the shampoo for developing hair color is used, not only the existing dyeing procedure that takes more than tens of minutes, but also an additional periodic treatment due to growing hair is not required, which makes it possible to greatly increase the convenience and develop a deep hair color in a short period of time while minimizing damage to the hair.

Further, another aspect of the present disclosure for achieving the above object provides a method of developing hair color comprising the step of cleansing the hair with the shampoo for developing hair color.

As used herein, the term “developing hair color” is as described above.

The step of cleansing the hair may be performed for 1 to 5 minutes, or 1 to 3 minutes, or 1 minute, but is not limited thereto.

According to the present disclosure, the composition for developing hair color comprising trihydroxybenzene produces the development of hair color in brown, dark brown, or black and has excellent color development enhancing power, color development lasting power, and anti-gray hair, without using amine compounds, irritating alkaline agents, and oxidizing agents.

Hereinafter, the present disclosure will be described in more detail with reference to examples. These examples are only for illustrating the present disclosure, and the scope of the present disclosure is not limited thereto.

### Example 1: Preparation of Composition for Developing Hair Color

Comparative Example (C. Ex.) and Preparation Examples 1 to 6 (Prep. Ex. 1 to 6) were prepared with the compositions shown in Table 1 below to confirm hair color development effects of various polyphenol compounds. The content unit of each component listed in Table 1 is % by weight.

TABLE 1

| | | Prep. | Prep. | Prep. | Prep. | Prep. | Prep. |
|---|---|---|---|---|---|---|---|
| | C. Ex. | Ex. 1 | Ex. 2 | Ex. 3 | Ex. 4 | Ex. 5 | Ex. 6 |
| Gallic acid | 0 | 1 | 0 | 0 | 0 | 0.5 | 0 |
| 3,4-dihydroxyhydro- | 0 | 0 | 1 | 0 | 0 | 0 | 1 |
| cinnamic acid | | | | | | | |
| Dopamine | 0 | 0 | 0 | 1 | 0 | 0 | 0 |
| 1,2,4- | 0 | 0 | 0 | 0 | 1 | 0 | 0 |
| trihydroxybenzene | | | | | | | |
| Polyethylenimine | 0 | 0 | 0 | 0 | 0 | 1 | 1 |
| Purified water | 100 | 99 | 99 | 99 | 99 | 98.5 | 98 |

### Experimental Example 1: Evaluation of Hair Color Development Effect of Composition for Developing Hair Color

In order to check the hair color development performance of the composition prepared in Example 1, the compositions of Comparative Example and Preparation Examples 1 to 6 were evenly applied to a portion of the bleached hair sample, respectively, and then left at room temperature for 3 minutes. After 3 minutes, washing was performed by rubbing in running water for 3 minutes, and residual moisture was removed to evaluate the hair color.

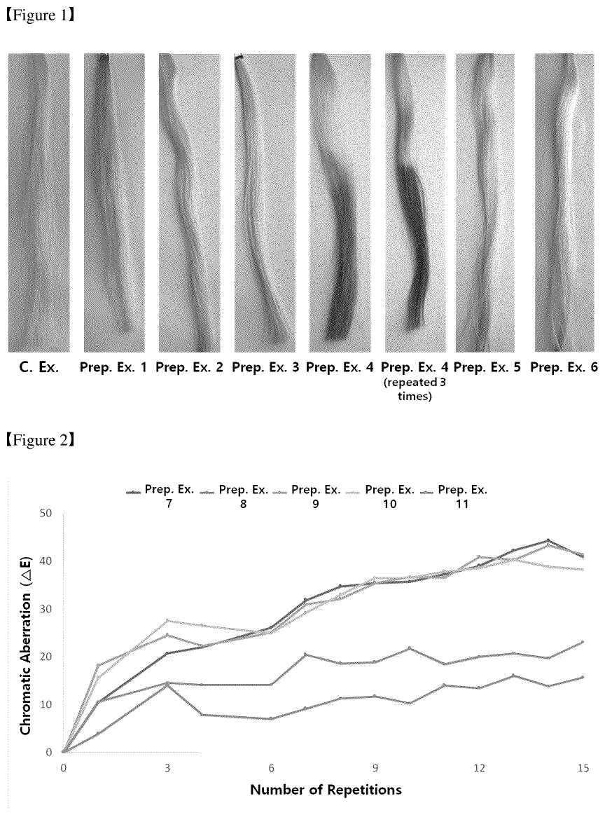

As a result, as shown in FIG. 1, Comparative Example and Preparation Examples 1 to 3, 5, and 6 did not show a clear hair color development effect. Preparation Example 4, which is an aqueous solution of 1,2,4-trihydroxybenzene, exhibited the most excellent color development effect. It was confirmed that when the application and washing of Preparation Example 4 were repeated 3 times, the hair developed a color more deeply.

### Example 2: Preparation of Composition for Developing Hair Color in Shampoo Formulation

In order to check the hair color development effect of the composition for developing hair color containing 1,2,4-trihydroxybenzene according to the present invention prepared as a shampoo formulation, the compositions of Preparation Examples 7 to 11 (Prep. Ex. 7) were prepared with the compositions shown in Table 2 below. The content unit of each component listed in Table 2 is % by weight.

TABLE 2

| | Prep. Ex. 7 | Prep. Ex. 8 | Prep. Ex. 9 | Prep. Ex. 10 | Prep. Ex. 11 |
|---|---|---|---|---|---|
| Shampoo | 85 | 85 | 85 | 85 | 85 |
| Caramel pigment | 0 | 1 | 1 | 10 | 10 |
| Mulberry (Morus sp.) | 0 | 0 | 1 | 0 | 0 |
| fruit extract | | | | | |
| Green tea extract | 1 | 1 | 1 | 1 | 1 |
| Natural complex | 1 | 1 | 1 | 1 | 1 |
| composition | | | | | |
| 1,2,4- | 1 | 0.1 | 1 | 1 | 0.1 |
| trihydroxybenzene | | | | | |
| Purified water | Balance | Balance | Balance | Balance | Balance |
| | (up to 100) | (up to 100) | (up to 100) | (up to 100) | (up to 100) |

The shampoo used an anionic surfactant, specifically disodium laureth sulfosuccinate, cocamidopropyl hydroxysultaine, glycerin, cocamide MIPA, lauryl glucoside, hexanediol, and purified water were mixed and used. Based on the total weight of the shampoo, about 30% by weight of disodium laurethsulfosuccinate, about 30% by weight of cocamidopropylhydroxysultaine, about 5% by weight of glycerin, about 1% by weight of cocamide MIPA, about 1% by weight of lauryl glucoside, and about 1% by weight of nucleic acid diol were used, and the remainder was purified water.

The natural complex composition of Table 2 was prepared in the following manner:

Washing Sesamum indicum seed extract 250 g, Morus nigra fruit extract 50 g, Tuber melanosporum extract 50 g, Nigella sativa seed extract 50 g, and Prunus serotina (wild cherry) fruit extract 250 g in purified water sufficiently; and extracting them under reduced pressure at 65 to 90° C. for 6 to 8 hours using 20 L of purified water, respectively, to yield extracts. Each hot water extract was mixed with gallic acid as shown in the composition ratios in Table 3 below to prepare the mixture.

TABLE 3

| Components | wt. % |
|---|---|
| Sesamum Indicum (Sesame) Seed Extract | 10 |
| Morus Nigra Fruit Extract | 10 |
| Tuber Melanosporum Extract | 10 |
| Nigella Sativa Seed Extract | 10 |
| Prunus Serotina (Wild Cherry) Fruit Extract | 10 |
| Gallic Acid | 0.001 |
| Purified water | Balance (up to 100) |

### Experimental Example 2: Evaluation of Hair Color Development Effect of Shampoo for Developing Hair Color

In order to check the hair color development performance of the shampoo for developing hair color prepared in Example 2, each of the shampoos of Preparation Examples 7 to 11 was evenly applied to a portion of bleached hair sample, rubbed at room temperature for 3 minutes while foaming, and then washed by rubbing in running water for 3 minutes. After repeating the foaming and washing process 15 times, the hair was dried with warm air to evaluate the hair color. Lab color values were measured using a colorimeter at the end of each cycle (shampooing and washing). Chromatic aberration before and after color development was evaluated and recorded by calculating using Equation 1 below.

ΔE=\sqrt{(L′−L)²+(a′−a)²+(b′−b)²} Equation 1

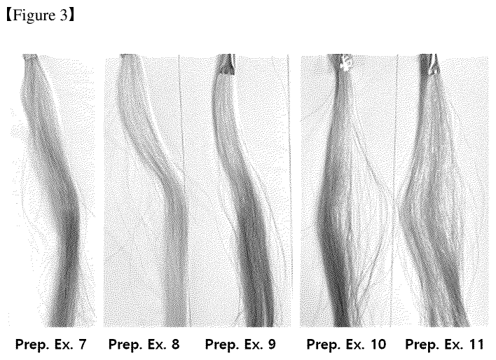

As shown in FIG. 2, we confirmed that the color was developed continuously during repeated washing 15 times for the change of chromatic aberration. The chromatic aberration was significantly increased compared to the hair before color development.

In addition, as shown in FIG. 3, after the repeated washing 15 times, we confirmed that the hair of the part to which the inventive shampoo for developing hair color was applied developed color brown.

1. A composition for developing hair color comprising trihydroxybenzene.

2. The composition for developing hair color of claim 1, wherein the developing hair color comprises hair browning or blackening, or preventing whitening.

3. The composition for developing hair color of claim 1, wherein the composition does not comprise an amine compound, an oxidizing agent, and an irritating alkaline agent.

4. The composition for developing hair color of claim 1, wherein the composition comprises 0.1 to 10% by weight of trihydroxybenzene based on the total weight of the composition.

5. The composition for developing hair color of claim 1, which further comprises a mixture of Sesamum indicum (sesame) seed extract, Morus nigra fruit extract, Tuber melanosporum extract, Nigella sativa seed extract, Prunus serotina (wild cherry) fruit extract, and gallic acid.

6. The composition for developing hair color of claim 1, which comprises at least one selected from the group consisting of caramel pigment, mulberry (Morus sp.) fruit extract, and green tea extract.

7. The composition for developing hair color of claim 1, wherein the composition is a shampoo, hair conditioner, hair treatment, hair lotion, hair gel, hair pack, hair cream, or hair essence formulation.

8. A method of developing hair color, which comprises applying the composition for developing hair color of claim 1 to the hair.

9. The method of developing hair color of claim 8, wherein after applying the composition for developing hair color to the hair, the method further comprises leaving the composition for 1 to 30 minutes.

10. A shampoo for developing hair color comprising the composition for developing hair color of any of claim 1.

11. The shampoo for developing hair color of claim 10, wherein the shampoo further comprises any one or more selected from the group consisting of surfactants, pH adjusters, swelling agents, buffers, preservatives, fragrances, moisturizers, viscosity modifiers, aliphatic alcohols, and vitamins.

12. A method of developing hair color, which comprises cleansing the hair with the shampoo for developing hair color of claim 10.

13. The method of developing hair color of claim 12, wherein the cleansing is performed for 1 to 5 minutes.
